(19) United States
(12) Patent Application Publication
(10) Pub. No.: US 2008/0097562 A1
(43) Pub. Date: Apr. 24, 2008

(54) System For Chemohyperthermia Treatment

(75) Inventor: Aik Ping Theodore TAN, Singapore (SG)

Correspondence Address: LAWRENCE Y.D. HO & ASSOCIATES PTE LTD, 30 BIDEFORD ROAD, #02-02, THONGSIA BUILDING, SINGAPORE, 229922, omitted

(73) Assignee: Dyamed Biotech Pte Ltd, Singapore (SG)

(21) Appl. No.: 11/551,235

(22) Filed: Oct. 19, 2006

(51) Int. Cl. A61F 7/00; A61F 7/12

(52) U.S. Cl. 607/105; 607/113

(57) ABSTRACT

The present invention provides a system for chemohyperthermia treatment. The chemohyperthermia treatment system comprises a reservoir for storing fluid; a heating/cooling system coupled to the reservoir so that the fluid can be transferred from the reservoir to the heating system, wherein the heating/cooling system comprises a heating/cooling exchange module having a channel within which the fluid can flow; and a plurality of peltier modules coupled to the heating/cooling module, wherein the plurality of peltier modules heat up the fluid flowing through the channel, and wherein in the cooling mode, the plurality of peltier modules cool the fluid flowing through the channel; a pumping means coupled to the heating/cooling system, wherein the pumping means pump the perfusion fluid from the reservoir to the heating/cooling system, thereby allowing the heating/cooling system to change the temperature of the fluid; at least one inflow catheter coupled to the pumping means, wherein the at least one inflow catheter delivers the heated/cooled fluid to an object; and at least one outflow catheter coupled to the reservoir, wherein the at least one outflow catheter drains the fluid from the object to the reservoir.

### FIELD OF THE INVENTION

The present invention generally relates to hyperthermia treatment, and more particularly to a chemohyperthermia system that provides stable heating of perfusion fluid and is compact.

### BACKGROUND OF THE INVENTION

Hyperthermia treatment generally refers to a process for treating certain illness by circulating a perfusate (perfusing fluid) in a body cavity of an object including human beings, where the circulated perfusate has been heated to a temperature that is higher than the normal body temperature of the object. One particular hyperthermia treatment is the chemohyperthermia treatment that is a fusion of chemotherapy and hyperthermia treatment. For chemohyperthermia treatment, the perfusing fluid in the body cavity is heated up to 45° C. to increase the susceptibility of cancer cells in the body cavity to the chemotheraputic agents. Chemohyperthermia treatment has been used as an adjunct therapy for cancer patients because it increases the survival rate of patients significantly and improves the quality of patients' life.

It has been established that chemohyperthermia treatment is very effective for the treatment of peritoneal cancer. One method of applying chemohyperthermia treatment is by perfusion of heated liquids (perfusate) into a body cavity of an object, which is known as intracavitary chemohyperthermia. An example of intracavitary chemohyperthermia is intraperitoneal chemohyperthermia (IPCH) treatment that circulates perfusate through the peritoneum. One known IPCH system is the ThermoChem HT-1000 from ViaCirq Inc. (US). The ThermoChem system is used to provide an adjunctive treatment that continuously circulates preheated perfusion fluid throughout the peritoneum, thereby increasing the temperature of the peritoneal cavity up to 45° C.

During chemohyperthermia treatment, it is critical to maintain the temperature of perfusate being introduced into a body cavity of an object with minimized heat spikes. In order to do so, the ThermoChem system uses a water bath heating system to provide stable and consistent heating of the perfusion fluid. The water bath heating system comprises a heat exchanger that uses a liquid-to-liquid heating interface to indirectly heat the perfusion fluid that is circulated into the patient's body. Although the ThermoChem system provides consistent heating of the perfusion fluid, it has certain drawbacks. For example, the water bath heating system requires additional components such as a water tank and water pump control modules. These additional components are usually housed in a separate compartment in order to prevent spillage into the main control system. As a result, the additional components and compartment increase the weight and profile of the ThermoChem system significantly. For example the ThermoChem system has a weight of 155 kg, height of 1.7 m, and width of 0.85 m.

Furthermore, some conventional IPCH systems use a close system, which is potentially dangerous to the patient. In a close system, the drainage of the perfusion fluid through the outflow catheter is achieved by the negative pressure created by roller pumps. As a result, the organ or tissue near the outflow catheter may suffer insidious damage

Therefore, there is an imperative need to have a chemohyperthermia system that provides stable heating of perfusion fluid and is compact.

### SUMMARY OF THE INVENTION

In one embodiment of the present invention, there is provided a system for chemohyperthermia treatment. The chemohyperthermia treatment system comprises a reservoir for storing fluid; a heating/cooling system coupled to the reservoir so that the fluid can be transferred from the reservoir to the heating system, wherein the heating/cooling system comprises a heating/cooling exchange module having a channel within which the fluid can flow; wherein the channel has an inlet for in-flowing the fluid and an outlet for out-flowing the fluid; and a plurality of peltier modules coupled to the heating/cooling module, wherein each of the plurality of peltier modules can operate in a heating mode or cooling mode independently; wherein in the heating mode, the plurality of peltier modules heat up the fluid flowing through the channel, and wherein in the cooling mode, the plurality of peltier modules cool the fluid flowing through the channel; a pumping means coupled to the heating/cooling system, wherein the pumping means pump the perfusion fluid from the reservoir to the heating/cooling system, thereby allowing the heating/cooling system to change the temperature of the fluid; at least one inflow catheter coupled to the pumping means, wherein the at least one inflow catheter delivers the heated/cooled fluid to an object; and at least one outflow catheter coupled to the reservoir, wherein the at least one outflow catheter drains the fluid from the object to the reservoir.

In another embodiment of the system, the heating/cooling exchange module comprises a body having a groove, and a conductor enclosing the groove to form the channel.

In another embodiment of the system, the pumping means further regulates the flow rate of the fluid in the system

In another embodiment of the system, it further comprises a tubing coupled between the pumping means and the reservoir, and a bypass switch configured to control the fluid flowing into either the at least one inflow catheter or the reservoir.

In another embodiment of the system, it further comprises a mini-reservoir coupled between the heating system and the pumping means to dampen the temperature of the heated fluid.

In another embodiment of the system, the reservoir comprises an air vent for releasing pressure so that the system can be an open or vented system.

In another embodiment of the system, the heating/cooling system further comprises a heating/cooling plate disposed between the plurality of peltier modules and the heating/cooling exchange module to transfer heat from the plurality of peltier modules to the heating/cooling exchange module or vice versa. In a further embodiment of the system, the heating/cooling system further comprises a heat sink coupled to the plurality of peltier modules to dissipate heat from the plurality of peltier modules. In another further embodiment of the system, the heating system further comprises a plurality of box fans coupled to the heat sink, wherein the plurality of box fans facilitates the heat sink in dissipating heat.

In another embodiment of the system, it further comprises a first pressure and temperature sensing probe coupled to the at least one inflow catheter for measuring the pressure and temperature of the fluid flowing into the object. In a further embodiment of the system, it further comprises a second pressure and temperature sensing probe coupled to the at least one outflow catheter for measuring the pressure and temperature of the perfusion fluid drained from the object. In another further embodiment of the system, it further comprises a third pressure and temperature sensing probe coupled to the heating system to measure the temperature of the fluid heated by the heating system. In yet another further embodiment of the system, it further comprises a level sensor coupled to the reservoir to detect the level of perfusion fluid in the reservoir, thereby preventing the perfusion fluid from over filling the reservoir or prevent the premature emptying of the perfusion fluid from the selected media.

In another embodiment of the system, it further comprises a computer system coupled to the level sensor and the first, second and third pressure and temperature sensing probes, wherein the computer system can be programmed to monitor the perfusion fluid level detected by the level sensor, and wherein the computer system can be programmed to monitor the temperature and pressure detected by the first, second and third pressure and temperature sensing probes. In a further embodiment of the system, the computer system comprises an interactive display means that enables a user to monitor and adjust the system parameters.

In another embodiment of the system, the pumping means comprises a plurality of roller pumps.

In another embodiment of the system, it further comprises self-contained fluid disposable drainage bag for collection of the fluid.

In another embodiment of the system, the chemohyperthermia treatment is an intracavitary one.

The chemohyperthermia treatment system of the present invention has many advantages. For example, the direct heating and monitoring system can be easily controlled and provides a consistent form of heating the perfusion fluid without any dangers of abrupt heat spikes. Furthermore, the direct heating system reduces the total amount of components for the system, thus resulting in a smaller weight and profile platform for the system. It is also important that the system does not physically contribute significantly to any form of tissue trauma to the patient while undergoing any established form of IPCH. Other advantages of this invention will be apparent with reference to the detailed description.

### BRIEF DESCRIPTION OF THE DRAWINGS

Preferred embodiments according to the present invention will now be described with reference to the Figures, in which like reference numerals denote like elements.

FIG. 1 is a block functional diagram of an intraperitoneal chemohyperthermia (IPCH) system in accordance with one embodiment of the present invention.

FIG. 2 is an exploded view of the direct heating/cooling system in accordance with one embodiment of the present invention.

FIG. 3 is an assembled view of the direct heating/cooling system in FIG. 2.

FIG. 4 is a cross-sectional view of the direct heating/cooling system looking from the line A-A.

### DETAILED DESCRIPTION OF THE INVENTION

The present invention may be understood more readily by reference to the following detailed description of certain embodiments of the invention.

The present invention provides a chemohyperthermia system that provides stable and consistent heating of the perfusion fluid. While the chemohyperthermia system will be illustrated by the exemplary intraperitoneal chemohyperthermia (IPCH) treatment in the description hereinafter, it is contemplated that the system may be used for other forms of treatment procedures within the bladder, lung and limb that require a perfusion fluid/body fluid to be heated up in a controlled manner.

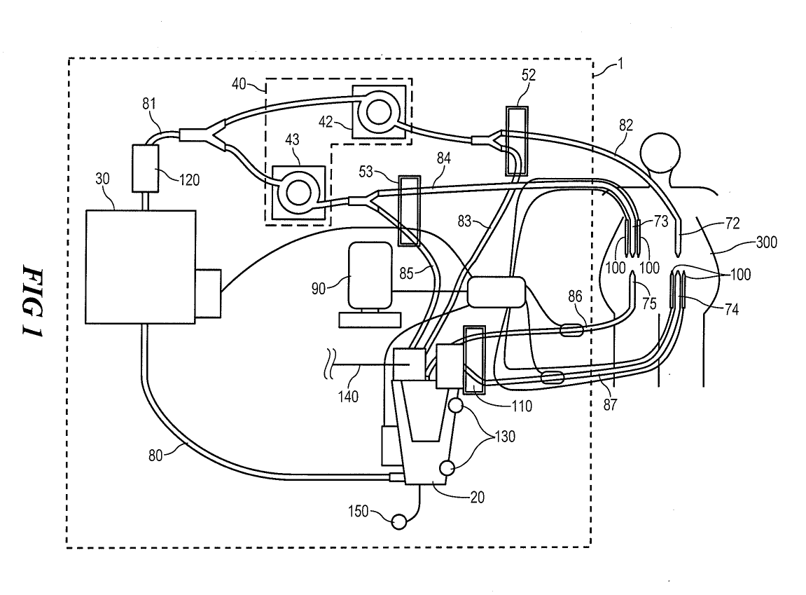

Referring to FIG. 1, there is provided a block functional diagram of an intraperitoneal chemohyperthermia (IPCH) system in accordance with one embodiment of the present invention. The IPCH system 1 comprises a reservoir 20, a direct heating/cooling system 30, a pump system 40, a plurality of bypass switches 52, 53, a plurality of temperature and pressure probes 100, and a plurality of inflow catheters 72, 73 and outflow catheters 74, 75.

The reservoir 20 functions as storage for perfusion fluid. The perfusion fluid that can be used in the present application is not limited any particular therapeutic reagents or composition. For example, the perfusion fluid may be standard peritoneal dialysis solution; and the choice of chemotherapeutic reagents can be determined by the physicians involved in performing the IPCH procedure. In addition, the reservoir 20 may comprise a filtration system for controlling the quality of the perfusion fluid flowing out from the reservoir 20. Furthermore, the reservoir 20 may have an air vent (not shown) for releasing pressure so that the IPCH system 1 can be an “open” or vented system. The reservoir 20 can be of any suitable configurations and dimensions.

The direct heating/cooling system 30 is coupled to the reservoir 20 via a tubing 80, wherein the tubing 80 serves as a channel for transferring perfusion fluid from the reservoir to the heating system 30. The direct heating/cooling system 30 is configured to ensure consistent regulation of temperature and rapid response to any necessary adjustments by using a solid-liquid heating/cooling method.

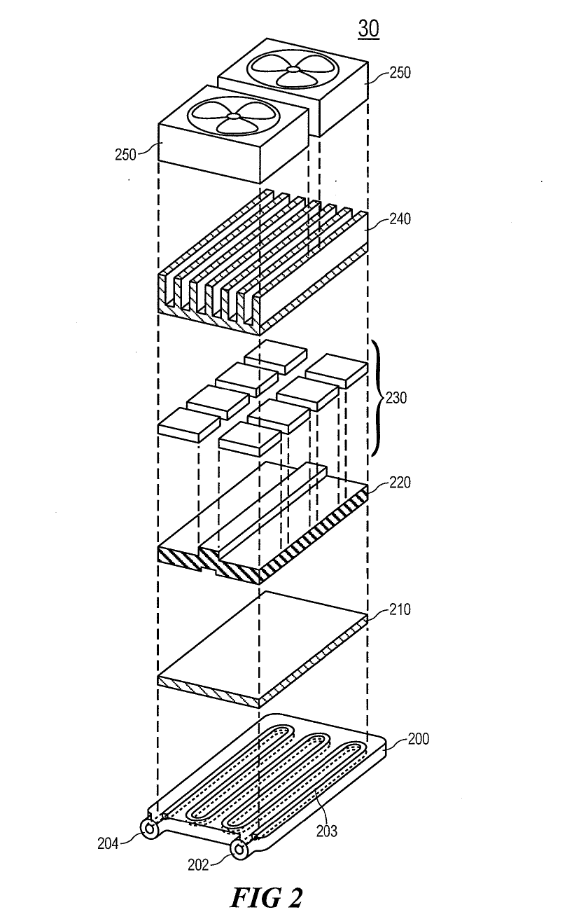

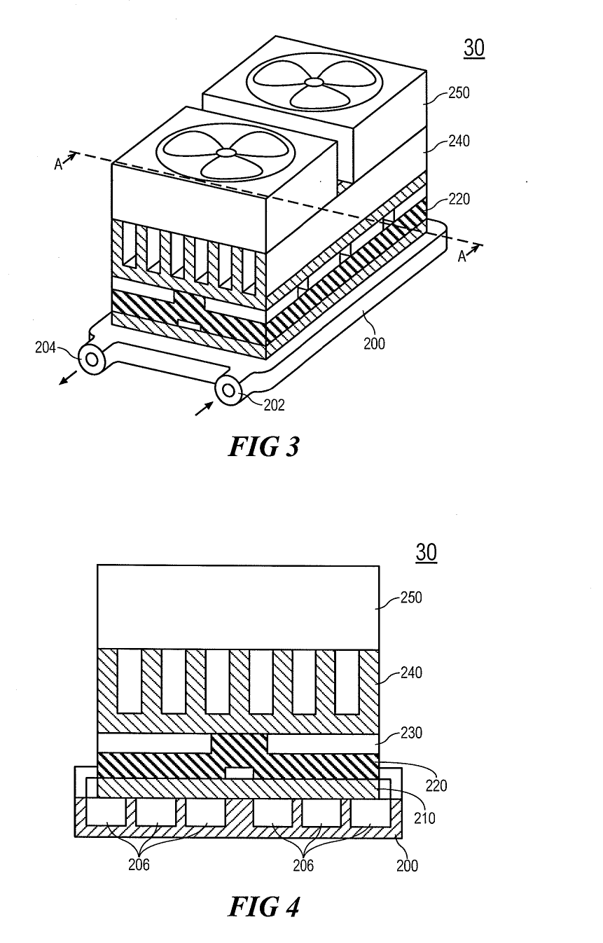

Referring to FIGS. 2-4, there are provided different views (exploded, assembled, or cross-sectional) of the direct heating/cooling system 30 in accordance with one embodiment of the present invention. As shown in FIG. 2, the direct heating/cooling system 30 comprises a heat exchanger 200, a conductor 210, a heating/cooling plate 220, a plurality of peltier modules 230, a heatsink 240, and a plurality of box fans 250.

The heat exchanger 200 comprises an inlet 202, a groove 203, and an outlet 204. The heat exchanger 200 can be made from any suitable materials including plastics or the like. The conductor 210 is coupled to the heat exchanger 200 to enclose the groove 203, thereby forming a fluid channel 206 as shown in FIG. 4. In operation, the inlet 202 receives perfusion fluid from the reservoir 20, wherein the perfusion fluid then flows through the channel 206 and exits from the outlet 204. The plurality of peltier modules 230 serve as the heating/cooling source for the direct heating/cooling system 30 and are coupled to the conductor 210 via the heating/cooling plate 220. In a heating mode, the plurality of peltier modules 230 heat up the heating/cooling plate 220. Then, the conductor 210 transfers the heat from the heating/cooling plate 220 to the perfusion fluid flowing in the channel 206, thereby heating up the perfusion fluid. The conductor 210 is preferably made from good heat conductivity materials such as aluminum. In a cooling mode, the plurality of peltier modules 230 cool down the heating/cooling plate 220, which in turn cools the conductor 210. As a result, the conductor 210 cools the perfusion fluid flowing through the channel 206.

In one embodiment, each of the plurality of peltier modules 230 has a first surface and second surface. While the first surfaces of the plurality of peltier modules 230 are coupled to the heating/cooling plate 220, the second surfaces of the plurality of peltier modules 230 are coupled to the heat sink 240. When in use, a voltage can be applied to the plurality of peltier modules 230 to achieve a temperature difference between the first surface and second surface of each peltier module 230. For example, the first surface can be hot and the second surface can be cold to achieve the heating mode. In the heating mode, the hot first surface of each peltier module 230 heats up the heating/cooling plate 220. As a result, the perfusion fluid flowing in the channel 206 is heated up as discussed above. In the cooling mode, the polarity of the voltage applied to the plurality of peltier modules 230 are simply reversed; thus the first surface of each peltier module 230 is cold and the second surface is hot. The cold first surface of each peltier module 230 cools down the heating/cooling plate 220 and prevents any potential heat spikes from occurring. As a result, the perfusion fluid flowing in the channel 206 is cooled down. In addition, the heat sink 240 dissipates the heat away from the hot second surface of each peltier module 230. The plurality of box fans 250 coupled to the heat sink 240 facilitates the dissipation of heat from the second surface of each peltier module 230.

The advantages of the direct heating/cooling system using the peltier module 230 are evident. For example, the direct heating/cooling system is a solid-state device with no moving parts, resulting in extreme reliability and little or no maintenance requirement. The peltier module 230 provides the desired, stable and consistent heating/cooling of the perfusion fluid without any dangers of sudden heat spikes by means of both hardware and software control. Furthermore, the use of the peltier module 230 reduces the total amount of components for the IPCH system 1 by eradicating the need for a bulky water tank and heat exchanger. Thus, the IPCH system 1 with its solid to liquid direct heating/cooling system 30 has a smaller weight and smaller profile platform as compared to the conventional systems using water tanks and heat exchangers.

Referring back to FIG. 1, the direct heating/cooling system 30 is coupled to the pump system 40 via the tubing 81. The pump system 40 controls the flow rate of the perfusion fluid for effective perfusion and dispersion. During operation, the pump system 40 pumps the perfusion fluid from the reservoir 20 to the direct heating/cooling system 30, wherein the perfusion fluid enters the inlet 202 of the heat exchanger 200 and flows through the channel 206. As the perfusion fluid is flowing through the channel 206, it is being heated or cooled by the plurality of peltier modules 230. Thereafter, the heated/cooled perfusion fluid exits the heat exchanger 200 from the outlet 204 and is transferred to the pump system 40. In a preferred embodiment, the pump system 40 comprises a first roller pump 42 and a second roller pump 43 that are configured to transfer the heated/cooled perfusion fluid from the direct heating/cooling system 30 into the peritoneal cavity 300. The two roller pumps 42, 43 provide a more effective and efficient perfusion fluid distribution into the patient's peritoneal cavity 300.

The first roller pump 42 is coupled to a first bypass switch 52, wherein the first bypass switch 52 is coupled to a first inflow catheter 72 via inflow tubing 82. Furthermore, the first bypass switch 52 is coupled to the reservoir 20 via inflow bypass tubing 83. The second roller pump 43 is coupled to a second bypass switch 53, wherein the second bypass switch 53 is coupled to a second inflow catheter 73 via tubing 84. Furthermore, the second bypass switch 53 is coupled to the reservoir 20 via second bypass tubing 85. The first and second inflow catheters (72, 73) deliver the heated/cooled perfusion fluid to the peritoneal cavity 300. Thereafter, the perfusion fluid is drained from the peritoneal cavity via outflow catheters 74, 75. The outflow catheters 74, 75 are coupled to the reservoir 20 via outflow tubings 86, 87, wherein the tubings 86, 87 transfer the perfusion fluid from the peritoneal cavity 300, referred herein as the peritoneal perfusate, to the reservoir 20. A gross pinch valve 110 can be coupled to the tubings 86, 87 to control the outflow of the peritoneal perfusate. The level of the perfusate in the reservoir 20 can be monitored by a high and low level sensor 130. Furthermore, the reservoir 20 acts as a gross filter to the peritoneal perfusate before transferring the filtered perfusion fluid to the direct heating/cooling system 30. In addition, perfusion fluid can be added to the reservoir 20 if necessary via an attachment tubing 140 connected to the inlet of the reservoir 20.

The first and second bypass switches (52, 53) allow the internal circulation of the perfusion fluid within the IPCH system 1. Internal circulation of the perfusion fluid allows it to be pre-heated to a desired temperature before being re-directed towards the inflow catheters (72,73) for circulation within the patient's peritoneal cavity 300. The operations of the first and second bypass switches (52, 53) are controlled by a computer system 90. When the computer system 90 detects a bypass event such as breach of safety levels, temperature, pressure, line occlusion or heat spike from the direct heating/cooling system 30, it activates the first and second bypass switches (52, 53) to open the tubings 83, 85 and close the tubings 82, 84, 86, 87. In this case, the “heat-spiked” perfusion fluid from the pumping system 40 is directed to the reservoir 20, thereby preventing the “heat-spiked” perfusion fluid from entering the patient's peritoneal cavity 300.

Still referring to FIG. 1, a secondary safety device (mini reservoir) 120 is disposed immediately after the direct heating/cooling system 30 to ensure that any “heat-spiked” perfusion fluid gets mixed adequately to bring down the temperature before it is transferred to the inflow catheters 72,73.

The drainage of the peritoneal perfusate from the peritoneal cavity 300 into the reservoir 20 may be achieved by the concept of gravitational siphoning or open system. Conventional IPCH systems uses closed system, wherein the closed system is not vented to equalized at atmospheric pressure from the patients' peritoneal cavity to the roller pump due to a lack of a vented reservoir. The advantage of an open system is that it helps to prevent negative pressure or sucking of organs and/or tissues located near the outflow catheters. As a result, tissue trauma due to the outflow catheters is reduced during the treatment process. In the present invention, the peritoneal perfusate is drained passively by the gravitational pull to the vented reservoir 20. As a result, the bare minimum negative suction is created by the siphoning effects of gravity and not by the uncontrolled actively created negative suction from the roller pumps.

Still referring to FIG. 1, the IPCH system 1 may further comprise a self-contained fluid disposable drainage bag system 150 that is used to collect fluid media in a safe manner for the operator. This minimizes the operator's risk of coming into contact with the contaminated chemical/biological fluid at the end of each treatment procedure. The inflow catheters (72, 73), outflow catheters (74, 75), and related perfusion apparels such as the tubings (80-87) are designed to be of sterile single use sets and can be made to be disposable at the end of each treatment procedure.

A plurality of pressure sensitive and temperature sensitive probes 100 are disposed at the inflow catheters (72, 73) and outflow catheters (74, 75) to monitor the operating pressure and temperatures so as to ensure patient safety. The plurality of probes 100 can be controlled by the computer system 90. Furthermore, the probe 100 can be disposed at the tubing 81 between the direct heating/cooling system 30 and the pumping system 40. The probe 100 is provided at the tubing 81 to ensure that the heated perfusion fluid is within safety limits. Another probe 100 can be deployed after the roller pumps (42, 43) to detect insidious or acute build up of pressure. Upon detection of this build up, the system software 90 will shut the roller pumps (42, 43) in order to prevent the bursting of tubings and thus keeping the integrity of the tubings for the continuation of the procedure upon physical rectification of the pressure build up by either the attending surgeon or perfusionist.

Temperature measurements probes 100 can be any available temperature measurement devices/technologies. In some embodiments, the temperature measurements probes 100 utilizes Resistance Temperature Detector (RTD) technology or Thermocouples Technology as a feedback temperature control.

The computer system 90 is coupled to the plurality of probes 100 for monitoring the operating temperatures and pressure. Furthermore, the computer system 90 can be coupled to the high and low level sensors 130 to monitor the level of perfusion fluid in the reservoir 20. Most importantly, the controller 90 is coupled to the direct heating/cooling system 30 for controlling the heating of the perfusion fluid. The controller 90 can be coupled to an interactive display interface (not shown) such as a touch screen monitor that enables the operator to monitor and adjust the system parameters accordingly to its embedded software control.

The system 1 may be operated by a perfusionist or other professionals who are trained in perfusion management as the characteristic of perfusion management techniques in open heart surgery is practically similar in requirements to that of an IPCH procedure. Typically, a perfusionist mainly uses the heart lung bypass machine that is ergonomically designed. The heart lung machine enables the perfusionist when seated on a stool to have a global view of the main circulatory components such as the pump, reservoir (this being the oxygenator) and inflow/outflow tubes. More importantly, the low profile of the machine enables the perfusionist to have an extended and unobstructed view of the operating procedure/setting, patient monitor and concurrent to manipulating the heart lung machinery. With lesser components, the IPCH system 1 tries to mimic the characteristic profile of that of the standard heart/lung setup in term of low height profile, ergonomic positioning of components, device mobility and ease of use. IPCH system 1 is low in height and weighs one third less than the other devices in the market.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A system for chemohyperthermia treatment comprising:
a reservoir for storing fluid;
a heating/cooling system coupled to the reservoir so that the fluid can be transferred from the reservoir to the heating system, wherein the heating/cooling system comprises:
a heating/cooling exchange module having a channel within which the fluid can flow; wherein the channel has an inlet for in-flowing the fluid and an outlet for out-flowing the fluid; and
a plurality of peltier modules coupled to the heating/cooling module, wherein each of the plurality of peltier modules can operate in a heating mode or cooling mode independently; wherein in the heating mode, the plurality of peltier modules heat up the fluid flowing through the channel, and wherein in the cooling mode, the plurality of peltier modules cool the fluid flowing through the channel;
a pumping means coupled to the heating/cooling system, wherein the pumping means pump the perfusion fluid from the reservoir to the heating/cooling system, thereby allowing the heating/cooling system to change the temperature of the fluid;
at least one inflow catheter coupled to the pumping means, wherein the at least one inflow catheter delivers the heated/cooled fluid to an object; and
at least one outflow catheter coupled to the reservoir, wherein the at least one outflow catheter drains the fluid from the object to the reservoir.

2. The system of claim 1, wherein the heating/cooling exchange module comprises a body having a groove, and a conductor enclosing the groove to form the channel.

3. The system of claim 1, wherein the pumping means further regulates the flow rate of the fluid in the system

4. The system of claim 1, further comprising a tubing coupled between the pumping means and the reservoir, and a bypass switch configured to control the fluid flowing into either the at least one inflow catheter or the reservoir.

5. The system of claim 1, further comprising a mini-reservoir coupled between the heating system and the pumping means to dampen the temperature of the heated fluid.

6. The system of claim 1, wherein the reservoir comprises an air vent for releasing pressure so that the system can be an open or vented system.

7. The system of claim 1, wherein the heating/cooling system further comprises a heating/cooling plate disposed between the plurality of peltier modules and the heating/cooling exchange module to transfer heat from the plurality of peltier modules to the heating/cooling exchange module or vice versa.

8. The system of claim 7, wherein the heating/cooling system further comprises a heat sink coupled to the plurality of peltier modules to dissipate heat from the plurality of peltier modules.

9. The system of claim 8, wherein the heating system further comprises a plurality of box fans coupled to the heat sink, wherein the plurality of box fans facilitates the heat sink in dissipating heat.

10. The system of claim 1, further comprising a first pressure and temperature sensing probe coupled to the at least one inflow catheter for measuring the pressure and temperature of the fluid flowing into the object.

11. The system of claim 10, further comprising a second pressure and temperature sensing probe coupled to the at least one outflow catheter for measuring the pressure and temperature of the perfusion fluid drained from the object.

12. The system of claim 11, further comprising a third pressure and temperature sensing probe coupled to the heating system to measure the temperature of the fluid heated by the heating system.

13. The system of claim 12, further comprising a level sensor coupled to the reservoir to detect the level of perfusion fluid in the reservoir, thereby preventing the perfusion fluid from over filling the reservoir or prevent the premature emptying of the perfusion fluid from the selected media.

14. The system of claim 13, further comprising a computer system coupled to the level sensor and the first, second and third pressure and temperature sensing probes, wherein the computer system can be programmed to monitor the perfusion fluid level detected by the level sensor, and wherein the computer system can be programmed to monitor the temperature and pressure detected by the first, second and third pressure and temperature sensing probes.

15. The system of claim 14 wherein the computer system comprises an interactive display means that enables a user to monitor and adjust the system parameters.

16. The system of claim 1, wherein the pumping means comprises a plurality of roller pumps.

17. The system of claim 1, further comprise self-contained fluid disposable drainage bag for collection of the fluid.

18. The system of claim 1, wherein the chemohyperthermia treatment is an intracavitary one.
